(19) United States
(12) Patent Application Publication
(10) Pub. No.: US 2014/0024558 A1
(43) Pub. Date: Jan. 23, 2014

(54) Method of Treatment

(71) Applicant: Glaxo Group Limited, Brentford (GB)

(72) Inventors: Kevin Lee, Stevenage (GB); David Francis Tough, Stevenage (GB); David Matthew Wilson, Stevenage (GB)

(73) Assignee: Glaxo Group Limited, Brentford (GB)

(21) Appl. No.: 14/032,249

(22) Filed: Sep. 20, 2013

Related U.S. Application Data
(62) Division of application No. 13/500,408, filed on Apr. 5, 2012.

(30) Foreign Application Priority Data
Oct. 27, 2009 (GB) 0818862.4

(51) Int. Cl. G01N 33/50

(52) U.S. Cl. CPC G01N 33/5008
USPC 506/11

(57) ABSTRACT

The present invention relates to methods for treatment or prevention of autoimmune and inflammatory diseases and conditions by inhibiting or modifying histone demethylation. In a further aspect the invention relates to a method for identifying agents useful in said methods of treatment. The invention particularly describes the role of certain histone demethylase enzymes in these diseases and conditions and their use as therapeutic and screening targets.

### CROSS-REFERENCE TO RELATED APPLICATIONS

This application is filed as a divisional application of U.S. Ser. No. 13/500,408, filed Apr. 5, 2012, which is a National Phase Application of International Application No. PCT/EP2010/066133, filed Oct. 26, 2010, which claims priority from 0918862.4, filed on Oct. 27, 2009 in the United Kingdom.

### FIELD OF THE INVENTION

The present invention is concerned with new methods of treatment. More particularly, the present invention relates to methods for treatment or prevention of autoimmune and inflammatory diseases and conditions by inhibiting or modifying histone demethylation. In a further aspect the invention relates to a method for identifying agents useful in said methods of treatment. The invention particularly describes the role of certain histone demethylase enzymes in these diseases and conditions and their use as therapeutic and screening targets.

### BACKGROUND OF THE INVENTION

Chromatin is the complex combination of DNA and protein that makes up chromosomes. It is found inside the nuclei of eukaryotic cells. It is divided between heterochromatin (condensed) and euchromatin (extended) forms. The major components of chromatin are DNA and proteins. Histones are the chief protein components of chromatin, acting as spools around which DNA winds. The basic building blocks of chromsatin are nucleosomes, each of which is composed of 146 base pairs of DNA wrapped around a histone octamer that consists of 2 copies of each H2A, H2B, H3 and H4. The functions of chromatin are to package DNA into a smaller volume to fit in the cell, to strengthen the DNA to allow mitosis and meiosis, and to serve as a mechanism to control expression and DNA replication. Chromatin contains genetic material serving as instructions to direct cell functions. Changes in chromatin structure are affected by chemical modifications of histone proteins such as methylation (DNA and proteins) and acetylation (proteins), and by non-histone, DNA-binding proteins. Several distinct classes of enzyme can modify histones at multiple sites.

Histone methylation is the modification of certain amino acids in a histone protein by addition of 1, 2 or 3 methyl groups. This methylation determines chromatin structure and regulated gene transcription. Histone methylation occur on both at lysine (K) and arginine (R) residues and has been linked to a number of cellular processes including DNA repair, replication, transcriptional activation and repression, for example, methylation of lysine 27 on histone H3 (H3-K27) is associated with epigenetically silenced (repressed) chromatin. Thus, regulation of the histone methylation status may control gene expression.

Histone methylation used to be regarded as a permanent/irreversible modification as compared to other histone modifications, however, with the discovery of histone demethylases, this is now considered to be a more dynamic modification. Levels of lysine methylation are known to change during processes such as transcriptional regulation. Therefore it was proposed that specific enzymatic activity might remove the methyl groups (1). Recent work has confirmed the existence of enzymatic demethylation and two separate mechanisms of lysine demethylation have been demonstrated: amine oxidation by LSD1 and hydroxylation by JmjC-domain containing proteins indicate these proteins as being novel histone modifying enzymes that can remove methyl groups on lysines (2, 3).

Lysine Specific Demethylase 1 (LSD1) is a flavin-dependent monoamine oxidase and can demethylate specific mono- and di-methylated lysines, histone 3, namely lysine 4 and 9 (H3K4 and H3K9). This enzyme cannot demethylate tri-methylated lysines.

The Jumonji protein is the founding member of a group of proteins characterised by a novel structural motif, the JmjC domain. The JmjC domain of several members of this family has been shown to possess lysine demethylation activity, which is dependent on iron and α-ketoglutarate as co-factors (4). JmjC domain-containing histone demethylases have been shown to demethylate mono-di, or trimethylated lysine.

Some JmjC domain-containing proteins, including histone demethylases have been implicated in tumorogenesis and thus have identified histone demethylases as targets of research for anti-cancer therapies. There is currently little information available concerning the role of histone demethylases in autoimmunity and inflammation, although one JmjC family demethylase, JMJD3, has been implicated in macrophage function (5-7). In these studies, JMJD3 expression was shown to increase in mouse macrophages in response to treatment with the bacterial product lipopolysaccharide (LPS) or the cytokine IL-4. In addition, knockdown or knockout of JMJD3 expression in the macrophages was associated with alterations in the expression of some LPS- or IL-4-induced genes. While these studies also reported the expression profiles of other demethylases in macrophages, the function of these demethylases was not investigated.

Jumonji-domain containing proteins including several histone demethylases have been implicated in tumorogenesis and thus histone demethylases have been identified as targets of research for anti-cancer therapies. (see for example WO2009/114011).

The present inventors have surprisingly found that histone demethylase enzymes, particularly JMJD2E, are involved in the inflammatory response. Inhibiting these targets, therefore, would provide a novel approach to the treatment of autoimmune and inflammatory diseases or conditions.

### SUMMARY OF THE INVENTION

The present invention is based on the observation that inhibiting the expression of certain histone demethylation enzymes, particularly JMJD2E, indicated benefits in autoimmunity and inflammation, for example, a reduction in pro-inflammatory cytokines and/or an increase in anti-inflammatory cytokines.

Thus in one aspect there is provided a method of treating autoimmune and inflammatory diseases and conditions which comprises modulating methylation on histones in a mammal. This modification may be effected in a variety of ways. In one aspect this is achieved using histone demethylase inhibitors.

In a further aspect there is provided a method of treatment of autoimmune and inflammatory diseases or condition in a mammal comprising administering a therapeutically effective amount of an inhibitor of JMJD2E.

In a further aspect there is provided the use of a modulator of histone demethylase activity in the manufacture of a medicament for the treatment of autoimmune and inflammatory diseases and conditions in a mammal. In a further aspect the modulator is a histone demethylase inhibitor.

In a further aspect the present invention provides the use of an inhibitor of JMJD2E in the manufacture of a medicament for the treatment of autoimmune and inflammatory diseases or conditions.

In a further aspect, there is provided a method of screening for a modulator of histone demethylase enzyme activity. In a further aspect the histone methylase is JMJD2E.

### DESCRIPTION OF DRAWINGS

FIG. 1. siRNAs targeting JMJD2E inhibit inflammatory cytokine production by curdlan-stimulated human dendritic cells. Monocyte-derived dendritic cells were transfected with 4 different siRNAs targeting JMJD2E or with a scrambled siRNA (All stars) as a negative control. The transfected dendritic cells were stimulated with curdlan, and the quantities of cytokines present in the medium 24 hours later were measured. The results show that all four siRNAs against JMJD2E inhibit the production of four inflammatory cytokines, IL-1β, TNF-α, IL-6 and IL-12. The data represent transfections done in triplicate for each siRNA (mean±SEM) for a single donor. Similar results were obtained in two additional donors.

FIG. 2. siRNAs targeting JMJD2E inhibit inflammatory cytokine production by LPS-stimulated human dendritic cells. Monocyte-derived dendritic cells were transfected with 4 different siRNAs targeting JMJD2E or with a scrambled siRNA as a negative control. The transfected dendritic cells were stimulated with LPS, and the quantities of TNF-α (A,C) and IL-6 (B,D) present in the medium 6 hours (A,B) or 24 hours (C,D) later were measured. (E) Cell viability measurements, taken at 24 hours after LPS stimulation, indicate that JMJD2E siRNA-induced reduction in cytokine production is not related to effects on cell viability. The data represent transfections done in sextuplicate for each siRNA (mean±SEM) for a single donor. Similar results were obtained in two additional donors.

### DETAILED DESCRIPTION OF THE INVENTION

As used herein, the term “histone demethylase inhibitor”, or “inhibitor” refers to any compound or treatment capable of inhibiting or reducing the expression or activity of a histone demethylase. The inhibitor is preferably selective against one or more histone demethylase enzymes with no direct activity as any other histone modifying enzymes.

Various histone demethylase enzymes have been identified and characterised. The following is particularly mentioned: —

### JMJD2E:

The nucleic acid sequence of human JMJD2E mRNA, including transcript variants, is provided by the following accession numbers: NM_{—}001146696, NM_{—}001146695, NM_{—}001146694, NM_{—}015061.

The amino acid sequence of human JMJD2E protein, including isoforms, is provided by the following accession numbers: NP_{—}055876, NP_{—}001140166, NP_{—}001140167, NP_{—}001140168.

These sequences are also available from other sources—it should be understood that the invention also pertains to novel variants and/or homologues of these specific sequences.

Within the scope of the present invention, an inhibitor of a human histone demethylase is preferably used, more particularly an inhibitor of JMJD2E, more particularly an inhibitor compound.

The inhibitor used can be any compound or treatment capable of inhibiting the expression of the histone demethylase, i.e. any compound or treatment that inhibits transcription of the gene, RNA maturation, RNA translation, post-translational modification of the histone demethylase enzyme protein, binding of the histone demethylase enzyme to a target and the like.

The inhibitor may be of varied nature and origin including natural origin [e.g. plant, animal, eukaryatic, bacterial, viral] or synthetic [particularly an organic, inorganic, synthetic or semi-synthetic molecule]. For example it can be a nucleic acid, a polypeptide, a protein, a peptide or a chemical compound.

In one aspect the inhibitor is an antisense nucleic acid capable of inhibiting transcription of the histone demethylase gene or translation of the corresponding messenger. The antisense nucleic acid can comprise all or part of the sequence of the histone demethylase gene, the histone demethylase messenger, or of a sequence that is complementary thereto. The antisense sequence can be a DNA, and RNA (e.g. siRNA), a ribozyme, etc. It may be single-stranded or double stranded. It can also be a RNA encoded by an antisense gene. When an antisense nucleic acid comprising part of the sequence of the gene or messenger under consideration is being used, it is preferred to use a part comprising at least 10 consecutive bases from the sequence, more preferably at least 15, in order to ensure specific hybridisation. In the case of an antisense oligonucleotide, it typically comprises less than 100 bases, for example in the order of 10 to 50 bases. This oligonucleotide can be modified to improve its stability, its nuclease resistance, its cell penetration, etc. Perfect complementarity between the sequence of the antisense molecule and that of the target gene or messenger is not required, but is generally preferred.

According to another embodiment, the inhibitor compound is a polypeptide. It may be, for example a peptide comprising a region of a histone demethylase sequence, and capable to antagonise its activity. A peptide advantageously comprises from 5 to 50 consecutive amino acids of the primary sequence of the demethylase under consideration, typically from 7 to 40. The polypeptide can also be an anti-histone demethylase antibody, or a fragment or derivative of such an antibody, for example a Fab fragment, a CDR region, or, more preferably, a single chain antibody (e.g. ScFv). Single chain antibodies are particularly advantageous insofar as they can act in a specific and intracellular fashion to modulate the activity of a target protein. Such antibodies, fragments, or derivatives can be produced by conventional techniques comprising immunising an animal and recovering the serum (polyclonal) or spleen cells (in order to produce hybridomas by fusion with appropriate cell lines).

Methods for producing polyclonal antibodies in various species are described in the prior art. Typically, the antigen is combined with an adjuvant (e.g. Freund's adjuvant) and administered to an animal, typically by subcutaneous injection. Repeated injections can be performed. Blood samples are collected and the immunoglobulin or serum is separated. Conventional method for producing monoclonal antibodies comprise immunising of an animal with an antigen, followed by recovery of spleen cells, which are then fused with immortalised cells, such as myeloma cells. The resulting hybridomas produce monoclonal antibodies and can be selected by limiting dilution in order to isolate individual clones. Fab or F(ab′)2 fragments can be produced by protease digestion, according to conventional techniques.

According to another embodiment, the inhibitor is a chemical compound, of natural or synthetic origin, particularly an organic or inorganic molecule, capable of modulating the expression or the activity of a histone demethylase. In a particular embodiment, the inhibitor is a small molecule.

As used herein, the “effective amount” means that amount of a drug or pharmaceutical agent that will elicit the biological or medical response of a tissue, system, animal or human that is being sought, for instance, by a researcher or clinician. Furthermore, the term “therapeutically amount” means any amount which as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. “Therapy” and “treatment” may include treatment and/or prophylaxis.

While it is possible that, for use in therapy, the inhibitor may be administered as the raw chemical, it is possible to present the active ingredient as a pharmaceutical composition. Accordingly, the invention further provides pharmaceutical compositions comprising an agent which JMJD2E and one or more pharmaceutically acceptable carriers, diluents, or excipients. The carrier(s), diluents(s) or excipient(s) must be acceptable in the sense of being compatible with the other ingredients of the composition and not deleterious to the recipient thereof. In accordance with another aspect of the invention there is also provided a process for the preparation of a pharmaceutical composition including the agent, or pharmaceutically acceptable salts thereof, with one or more pharmaceutically acceptable carriers, diluents or excipients. The pharmaceutical composition can be for use in the treatment and/or prophylaxis of any of the conditions described herein.

Pharmaceutical compositions may be presented in unit dose forms containing a predetermined amount of active ingredient per unit dose. Preferred unit dosage compositions are those containing a daily dose or sub-dose, or an appropriate fraction thereof, of an active ingredient. Such unit doses may therefore be administered once or more than once a day. Such pharmaceutical compositions may be prepared by any of the methods well known in the pharmacy art.

Pharmaceutical compositions may be adapted for administration by any appropriate route, for example by the oral (including buccal or sublingual), rectal, inhaled, intranasal, topical (including buccal, sublingual or transdermal), vaginal or parenteral (including subcutaneous, intramuscular, intravenous or intradermal) route. Such compositions may be prepared by any method known in the art of pharmacy, for example by bringing into association the active ingredient with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by reducing the compound to a suitable fine size and mixing with a similarly prepared pharmaceutical carrier such as an edible carbohydrate, as, for example, starch or mannitol. Flavouring, preservative, dispersing and colouring agent can also be present.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, glidants, lubricants, sweetening agents, flavours, disintegrating agents and colouring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavoured aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit compositions for oral administration can be microencapsulated. The composition can also be prepared to prolong or sustain the release as for example by coating or embedding particulate material in polymers, wax or the like.

The compounds of the invention may also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Pharmaceutical compositions adapted for transdermal administration may be presented as discrete patches intended to remain in intimate contact with the epidermis of the recipient for a prolonged period of time.

Pharmaceutical compositions adapted for topical administration may be formulated as ointments, creams, suspensions, lotions, powders, solutions, pastes, gels, sprays, aerosols or oils.

For treatments of the eye or other external tissues, for example mouth and skin, the compositions are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical compositions adapted for topical administrations to the eye include eye drops wherein the active ingredient is dissolved or suspended in a suitable carrier, especially an aqueous solvent.

Pharmaceutical compositions adapted for topical administration in the mouth include lozenges, pastilles and mouth washes.

Pharmaceutical compositions adapted for rectal administration may be presented as suppositories or as enemas.

Dosage forms for nasal or inhaled administration may conveniently be formulated as aerosols, solutions, suspensions drops, gels or dry powders.

For compositions suitable and/or adapted for inhaled administration, it is preferred that the agent is in a particle-size-reduced form, and more preferably the size-reduced form is obtained or obtainable by micronisation. The preferable particle size of the size-reduced (e.g. micronised) compound or salt or solvate is defined by a D50 value of about 0.5 to about 10 microns (for example as measured using laser diffraction). Compositions adapted for administration by inhalation include the particle dusts or mists. Suitable compositions wherein the carrier is a liquid for administration as a nasal spray or drops include aqueous or oil solutions/suspensions of the active ingredient which may be generated by means of various types of metered dose pressurised aerosols, nebulizers or insufflators.

Aerosol formulations, e.g. for inhaled administration, can comprise a solution or fine suspension of the agent in a pharmaceutically acceptable aqueous or non-aqueous solvent. Aerosol formulations can be presented in single or multidose quantities in sterile form in a sealed container, which can take the form of a cartridge or refill for use with an atomising device or inhaler. Alternatively the sealed container may be a unitary dispensing device such as a single dose nasal inhaler or an aerosol dispenser fitted with a metering valve (metered dose inhaler) which is intended for disposal once the contents of the container have been exhausted.

Where the dosage form comprises an aerosol dispenser, it preferably contains a suitable propellant under pressure such as compressed air, carbon dioxide or an organic propellant such as a hydrofluorocarbon (HFC). Suitable HFC propellants include 1,1,1,2,3,3,3-heptafluoropropane and 1,1,1,2-tetrafluoroethane. The aerosol dosage forms can also take the form of a pump-atomiser. The pressurised aerosol may contain a solution or a suspension of the active compound. This may require the incorporation of additional excipients e.g. co-solvents and/or surfactants to improve the dispersion characteristics and homogeneity of suspension formulations. Solution formulations may also require the addition of co-solvents such as ethanol. Other excipient modifiers may also be incorporated to improve, for example, the stability and/or taste and/or fine particle mass characteristics (amount and/or profile) of the formulation.

For pharmaceutical compositions suitable and/or adapted for inhaled administration, the pharmaceutical composition may be a dry powder inhalable composition. Such a composition can comprise a powder base such as lactose, glucose, trehalose, mannitol or starch, the agent, (preferably in particle-size-reduced form, e.g. in micronised form), and optionally a performance modifier such as L-leucine or another amino acid, cellobiose octaacetate and/or metals salts of stearic acid such as magnesium or calcium stearate.

Aerosol formulations are preferably arranged so that each metered dose or “puff” of aerosol contains a particular amount of a compound of the invention. Administration may be once daily or several times daily, for example 2, 3 4 or 8 times, giving for example 1, 2 or 3 doses each time. The overall daily dose and the metered dose delivered by capsules and cartridges in an inhaler or insufflator will generally be double those with aerosol formulations.

Pharmaceutical compositions adapted for vaginal administration may be presented as pessaries, tampons, creams, gels, pastes, foams or spray formulations.

Pharmaceutical compositions adapted for parental administration include aqueous and non-aqueous sterile injection solutions which may contain anti-oxidants, buffers, bacteriostats and solutes which render the composition isotonic with the blood of the intended recipient; and aqueous and non-aqueous sterile suspensions which may include suspending agents and thickening agents. The compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the compositions may include other agents conventional in the art having regard to the type of formulation in question, for example those suitable for oral administration may include flavouring agents.

Antisense or RNA interference molecules may be administered to the mammal in need thereof. Alternatively, constructs including the same may be administered. Such molecules and constructs can be used to interfere with the expression of the protein of interest, e.g., histone demethylase and as such, modify histone demethylation. Typically delivery is by means known in the art.

Antisense or RNA interference molecules can be delivered in vitro to cells or in vivo, e.g., to tumors of a mammal. Nodes of delivery can be used without limitations, including: intravenous, intramuscular, intraperitoneal, intra-arterial, local delivery during surgery, endoscopic, subcutaneous, and per os. Vectors can be selected for desirable properties for any particular application. Vectors can be viral or plasmid. Adenoviral vectors are useful in this regard. Tissue-specific, cell-type specific, or otherwise regulatable promoters can be used to control the transcription of the inhibitory polynucleotide molecules. Non-viral carriers such as liposomes or nanospheres can also be used.

A therapeutically effective amount of the agent will depend upon a number of factors including, for example, the age and weight of the subject, the precise condition requiring treatment and its severity, the nature of the formulation, and the route of administration, and will ultimately be at the discretion of the attendant physician or veterinarian. In particular, the subject to be treated is a mammal, particularly a human.

The agent may be administered in a daily dose. This amount may be given in a single dose per day or more usually in a number (such as two, three, four, five or six) of sub-doses per day such that the total daily dose is the same.

The agent may be employed alone or in combination with other therapeutic agents.

The agent for use in the present invention may be used in combination with or include one or more other therapeutic agents and may be administered either sequentially or simultaneously by any convenient route in separate or combined pharmaceutical compositions.

The agent and pharmaceutical compositions contain the invention may be used in combination with or include one or more other therapeutic agents, for example selected from NSAIDS, corticosteroids, COX-2 inhibitors, cytokine inhibitors, anti-TNF agents, inhibitors oncostatin M, anti-malarials, immunsuppressive and cytostatics

### Methods of Treatment and Diseases

Provided herein are methods of treatment or prevention of autoimmune and inflammatory conditions and diseases that can be improved by modulating the methylation status of histones, and thereby, e.g., modulate the level of expression of methylation activated and methylation repressed target genes. A method may comprise administering to a subject, e.g. a subject in need thereof, a therapeutically effective amount of an agent described herein.

Thus in one aspect there is provided the use of a histone demethylase inhibitor in the manufacture of a medicament for treating autoimmune and inflammatory diseases or conditions.

In a further aspect there is provided a method of treatment of autoimmune and inflammatory diseases or condition in a mammal comprising administering a therapeutically effective amount of a histone demethylase inhibitor.

In one aspect the histone demethylase is JMJD2E.

In one aspect the inhibitor inhibits JMJD2E.

Based at least on the fact that increased histone methylation has been found to be associated with inflammation, a method for treating inflammation in a subject may comprise administering to the subject a therapeutically effective amount of one or more agents that decrease methylation or restores methylation to its level in corresponding normal cells.

Inflammation represents a group of vascular, cellular and neurological responses to trauma. Inflammation can be characterised as the movement of inflammatory cells such as monocytes, neutrophils and granulocytes into the tissues. This is usually associated with reduced endothelial barrier function and oedema into the tissues. Inflammation can be classified as either acute or chronic. Acute inflammation is the initial response of the body to harmful stimuli and is achieved by the increased movement of plasma and leukocytes from the blood into the injured tissues. A cascade of biochemical event propagates and matures the inflammatory response, involving the local vascular system, the immune system, and various cells within the injured tissue. Prolonged inflammation, known as chronic inflammation, leads to a progressive shift in the type of cells which are present at the site of inflammation and is characterised by simultaneous destruction and healing of the tissue from the inflammatory process.

When occurring as part of an immune response to infection or as an acute response to trauma, inflammation can be beneficial and is normally self-limiting. However, inflammation can be detrimental under various conditions. This includes the production of excessive inflammation in response to infectious agents, which can lead to significant organ damage and death (for example, in the setting of sepsis). Moreover, chronic inflammation is generally deleterious and is at the root of numerous chronic diseases, causing severe and irreversible damage to tissues. In such settings, the immune response is often directed against self-tissues (autoimmunity), although chronic responses to foreign entities can also lead to bystander damage to self tissues.

The aim of anti-inflammatory therapy is therefore to reduce this inflammation, to inhibit autoimmunity when present and to allow for the physiological process or healing and tissue repair to progress.

The compounds of the invention may be used to treat inflammation of any tissue and organs of the body, including musculoskeletal inflammation, vascular inflammation, neural inflammation, digestive system inflammation, ocular inflammation, inflammation of the reproductive system, and other inflammation, as exemplified below.

Musculoskeletal inflammation refers to any inflammatory condition of the musculoskeletal system, particularly those conditions affecting skeletal joints, including joints of the hand, wrist, elbow, shoulder, jaw, spine, neck, hip, knew, ankle, and foot, and conditions affecting tissues connecting muscles to bones such as tendons. Examples of musculoskeletal inflammation which may be treated with compounds of the invention include arthritis (including, for example, osteoarthritis, rheumatoid arthritis, psoriatic arthritis, ankylosing spondylitis, acute and chronic infectious arthritis, arthritis associated with gout and pseudogout, and juvenile idiopathic arthritis), tendonitis, synovitis, tenosynovitis, bursitis, fibrositis (fibromyalgia), epicondylitis, myositis, and osteitis (including, for example, Paget's disease, osteitis pubis, and osteitis fibrosa cystic).

Ocular inflammation refers to inflammation of any structure of the eye, including the eye lids. Examples of ocular inflammation which may be treated with the compounds of the invention include blepharitis, blepharochalasis, conjunctivitis, dacryoadenitis, keratitis, keratoconjunctivitis sicca (dry eye), scleritis, trichiasis, and uveitis.

Examples of inflammation of the nervous system which may be treated with the compounds of the invention include encephalitis, Guillain-Barre syndrome, meningitis, neuromyotonia, narcolepsy, multiple sclerosis, myelitis and schizophrenia.

Examples of inflammation of the vasculature or lymphatic system which may be treated with the compounds of the invention include arthrosclerosis, arthritis, phlebitis, vasculitis, and lymphangitis.

Examples of inflammatory conditions of the digestive system which may be treated with the compounds of the invention include cholangitis, cholecystitis, enteritis, enterocolitis, gastritis, gastroenteritis, inflammatory bowel disease (such as Crohn's disease and ulcerative colitis), ileitis, and proctitis.

Examples of inflammatory conditions of the reproductive system which may be treated with the compounds of the invention include cervicitis, chorioamnionitis, endometritis, epididymitis, omphalitis, oophoritis, orchitis, salpingitis, tubo-ovarian abscess, urethritis, vaginitis, vulvitis, and vulvodynia.

The compounds of the invention may be used to treat autoimmune conditions having an inflammatory component. Such conditions include acute disseminated alopecia universalise, Behcet's disease, Chagas' disease, chronic fatigue syndrome, dysautonomia, encephalomyelitis, ankylosing spondylitis, aplastic anemia, hidradenitis suppurativa, autoimmune hepatitis, autoimmune oophoritis, celiac disease, Crohn's disease, diabetes mellitus type 1, giant cell arteritis, goodpasture's syndrome, Grave's disease, Guillain-Barre syndrome, Hashimoto's disease, Henoch-Schönlein purpura, Kawasaki's disease, lupus erythematosus, microscopic colitis, microscopic polyarteritis, mixed connective tissue disease, multiple sclerosis, myasthenia gravis, opsocionus myoclonus syndrome, optic neuritis, ord's thyroiditis, pemphigus, polyarteritis nodosa, polymyalgia, rheumatoid arthritis, Reiter's syndrome, Sjogren's syndrome, temporal arteritis, Wegener's granulomatosis, warm autoimmune haemolytic anemia, interstitial cystitis, lyme disease, morphea, psoriasis, sarcoidosis, scleroderma, ulcerative colitis, and vitiligo.

The compounds of the invention may be used to treat T-cell mediated hypersensitivity diseases having an inflammatory component. Such conditions include contact hypersensitivity, contact dermatitis (including that due to poison ivy), uticaria, skin allergies, respiratory allergies (hayfever, allergic rhinitis) and gluten-sensitive enteropathy (Celliac disease).

Other inflammatory conditions which may be treated with the compounds of the invention include, for example, appendicitis, dermatitis, dermatomyositis, endocarditis, fibrositis, gingivitis, glossitis, hepatitis, hidradenitis suppurativa, iritis, laryngitis, mastitis, myocarditis, nephritis, otitis, pancreatitis, parotitis, percarditis, peritonoitis, pharyngitis, pleuritis, pneumonitis, prostatistis, pyelonephritis, and stomatisi, transplant rejection (involving organs such as kidney, liver, heart, lung, pancreas (e.g., islet cells), bone marrow, cornea, small bowel, skin allografts, skin homografts, and heart valve xengrafts, sewrum sickness, and graft vs host disease), acute pancreatitis, chronic pancreatitis, acute respiratory distress syndrome, Sexary's syndrome, congenital adrenal hyperplasis, nonsuppurative thyroiditis, hypercalcemia associated with cancer, pemphigus, bullous dermatitis herpetiformis, severe erythema multiforme, exfoliative dermatitis, seborrheic dermatitis, seasonal or perennial allergic rhinitis, bronchial asthma, contact dermatitis, astopic dermatitis, drug hypersensistivity reactions, allergic conjunctivitis, keratitis, herpes zoster ophthalmicus, iritis and oiridocyclitis, chorioretinitis, optic neuritis, symptomatic sarcoidosis, fulminating or disseminated pulmonary tuberculosis chemotherapy, idiopathic thrombocytopenic purpura in adults, secondary thrombocytopenia in adults, acquired (autroimmine) haemolytic anemia, leukaemia and lymphomas in adults, acute leukaemia of childhood, regional enteritis, autoimmune vasculitis, multiple sclerosis, chronic obstructive pulmonary disease, solid organ transplant rejection, sepsis. Preferred treatments include treatment of transplant rejection, rheumatoid arthritis, psoriatic arthritis, multiple sclerosis, Type 1 diabetes, asthma, inflammatory bowel disease, systemic lupus erythematosis, psoriasis, chronic pulmonary disease, and inflammation accompanying infectious conditions (e.g., sepsis).

The methods of treatment and uses of the invention can be used in mammals, particularly in humans.

The present invention also provides a method for identifying agents which may be candidate compounds for the treatment of autoimmune and inflammatory diseases or conditions comprising determining whether a compound is capable of inhibiting JMJD2E.

### Screening Methods

The present invention proposes, for the first time that histone demethylases, particularly JMJD2E, as therapeutic targets for the treatment of autoimmune inflammatory diseases and conditions. Thus the present invention provides new targets for the identification, validation, selection and optimisation of active compounds on the basis of their ability to modulate the expression or activity of histone demethylase, particularly JMJD2E.

The assays (screening methods) may be performed in a cell-based system, an animal system or by a cell free system. Such techniques will be apparent to a person skilled in the art and may be based on a measure of interaction [e.g. binding, displacement or competition assays) or a measure of a function of activity, transcription and the like.

Also provided herein are screening methods for identifying agents that modulate methylation of histones as being potentially useful in the treatment of prevention of inflammation. One method involves screening for an inhibitor of histone demethylase activity, including the steps of contacting a histone peptide with a histone demethylase protein in the presence and in the absence of a test substance, determining the methylation status of the histone and identifying a test substance as an enhancer of histone demethylase activity if less mono-, di- or trimethylated histone is found in the presence than in the absence of the test substance, and identifying a test substance as an inhibitor of histone demethylase protein activity if more mono-, di- or trimethylated histone is found in the presence than in the absence of the test substance. Test agents (or substances) for screening as inhibitors or enhancers of the demethylase enzymes can be from any source known in the art. They can be natural products, purified or mixtures, synthetic compounds, members of compound libraries, etc. The test substances can be selected from those that have previously identified to have biological or drug activity or from those that have not.

In a further aspect the method of screening for an inhibitor of histone demethylase protein includes a binding assay. Thus a compound which inhibits the binding of a histone demethylase protein to its substrate can be identified in competition or direct binding assays. Both direct and competition binding assay formats are similar to the formats used in immunoassays and receptor binding assays and will be generally known to a person skilled in the art.

In one aspect the histone demethylase protein is JMJD2E.

The following examples are set forth to illustrate the effectiveness of the approach described in the present invention and to further exemplify particular applications of general processes described above. Accordingly, the following Example section is in no way intended to limit the scope of the invention contemplated herein.

To investigate whether histone demethylases might represent targets for treatment of autoimmune and inflammatory diseases and conditions, we tested, individually, the function of all histone demethylases and JmjC family proteins in immune cells using small inhibitory RNAs (siRNAs). siRNAs bind specifically to mRNA transcripts which bear complementary nucleotide sequences and subsequently reduce expression of the protein encoded by that specific mRNA. siRNA “knockdown” is a well established procedure to investigate the function of a specific gene product. We used an siRNA library targeting 34 known histone demethylases and JmjC proteins (Table 1).

TABLE 1
List of demethylase targets in library

| Gene | |
|---|---|
| JHDM1D | LOC390245 (JMJD2E) |
| JMJD2E | JMJD1B |
| JMJD2A | JMJD3 |
| HIF1AN | JMJD4 |
| FBXL11 | JARID2 |
| JMJD1C | PHF2 |
| JMJD2B | JMJD5 |
| JARID1C | HSPBAP1 |
| JARID1A | JARID1B |
| LOC339123 | HR |
| JMJD1A | UTY |
| AOF1 | UTX |
| JARID1D | JMJD2D |
| C14orf169 | FBXL10 |
| AOF2 | C2orf60 |
| MINA | PHF8 |
| LOC100137047- | |
| PLA2G4B | |
| JMJD6 | |

Since a number of disparate factors can influence the efficacy of knockdown by an siRNA, and advanced algorithms capable of accurately predicting efficacious siRNAs have not yet been developed, it has been recommended to use a minimum of three to four siRNAs against a given target when conducting a screen (9). We used four distinct siRNAs targeting each gene, including JMJD2E, for our studies.

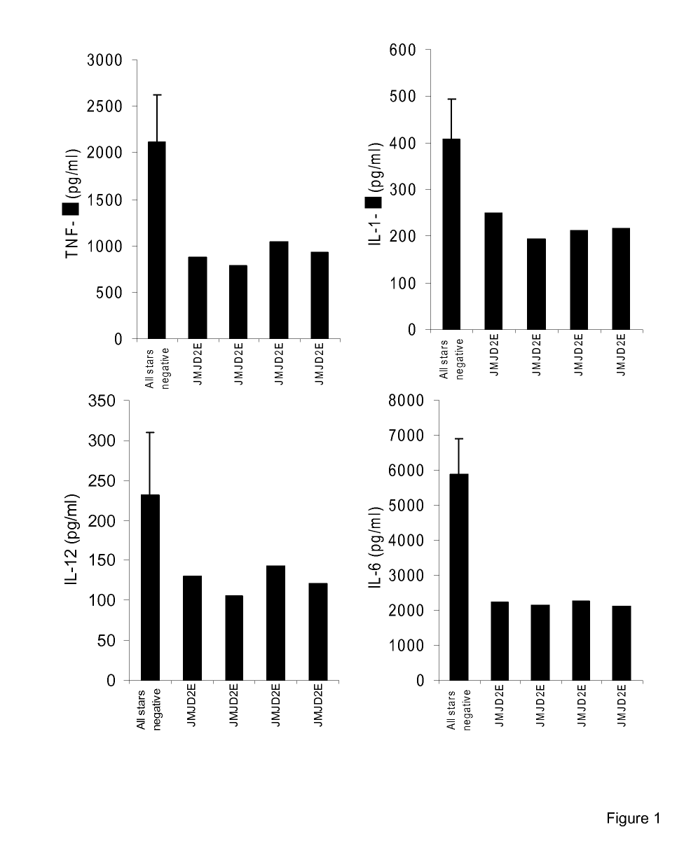

An siRNA screen was conducted in primary human monocyte-derived DCs. Subsequently, the cells were stimulated by treatment with curdlan and cultured for twenty-four hours. The quantities of four pro-inflammatory cytokines, IL-1β, IL-12, IL-6 and TNF-α, and the anti-inflammatory cytokine IL-10, were measured in the medium. siRNAs directed against JMJD2E, were found to inhibit significantly the production of all four pro-inflammatory cytokines (FIG. 1). All four siRNAs against JMJD2E produced similar inhibitory effects.

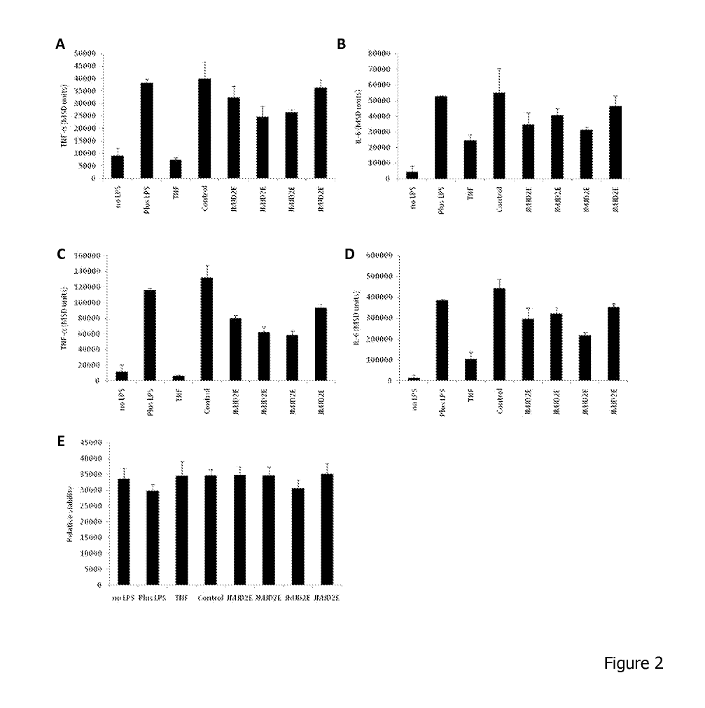

siRNAs targeting JMJD2E were also tested for their ability to inhibit cytokine production by DCs activated with a different stimulus, lipopolysaccharide (LPS). As shown in FIG. 2, TNF-α and IL-6 production were reduced in DCs treated with JMJD2E siRNAs compared to control cells at 6 hours (FIGS. 2A,B) and 24 hours (FIGS. 2C,D) after stimulating the cells with LPS. Effects on IL-1β and IL-12 production could not be assessed as these cytokines were poorly induced by LPS. Notably, cell viability was not reduced by the JMJD2E siRNAs, indicating that effects on cytokines were not due to general effects on cell viability (FIG. 2E).

In summary, these data demonstrate an unexpected role for JMJD2E in the inflammatory immune response, particularly in the production of inflammatory cytokines by DCs in response to various stimuli. Inhibiting the expression and/or activity of JMJD2E represents a novel approach to the treatment of autoimmune and inflammatory diseases and conditions.

### Methods:

### DC Transfection and Activation:

DCs were generated as described above and transfected using the monocyte cell nucleofecter kit (Amaxa—VHPA-1007). siRNA reagents were pre-plated into 96 well U′bottom plates such that a final concentration of 2 μM would be achieved. Nucleofecter buffer was made up according to the manufacturers protocol. DCs were centrifuged at 1500 rpm for 10 minutes and all growth media removed. Nucleofecter buffer (plus supplement) was added to the cells such that each 20 μl contained 100,000 cells. Cells were then added to the siRNA reagents in the U′bottom plates. 20 μL aliquots of cells/siRNAs were carefully added to each well of a 96 well nucleofecter plate. The plate was placed into the Amaxa nucleofector and the monocyte program EA-100 applied to all wells. After removal of the Amaxa plate, 100 μl of pre-warmed RPMI medium (10% HI FBS, Penicillin/Streptomycin/L-Glutamine PS) was added to each well. Cells were immediately removed from the Amaxa plate wells (100 μl was removed from the plate to ensure a consistent volume recovered) and added to a second U′bottom plate containing an additional 100 μl of pre-warmed media. After incubation for 24 hours at 37° C., curdlan was added at 100 μg/ml or LPS was added at 100 ng/ml. Supernatants were collected 6-24 hours later and cytokines were measured using MSD plates and read in a MSD Sector 6000 plate reader. Viability was measured using the CellTiter-Blue® Cell Viability Assay (Promega).

### Screen Hit Calling

Using the response library data, a robust mean and standard deviation was calculated on a plate by plate basis for each donor for each reporter cytokine. Two cut offs were also calculated to find the extreme 10% of the data in each tail. Any response greater than robust mean+1.3×robust standard deviation was deemed an increasing hit and any response less than robust mean−1.3×robust standard deviation was deemed an inhibited hit. The analysis used log responses, using log to the base 10.

### Materials:

### Miltenyi Buffer: PBS w/o Ca and Mg, 0.5% BSA, 5 mM EDTA

Miltenyi (MACS) CD14 Beads: CD14 MicroBeads, human 2 ml, contains 0.1% BSA, 0.05% Azide (in cold room).

### BSA: Albumin, Bovine Fraction V Powder (Sigma A-1933)

EDTA: Ethylenediaminetetraacetic acid (Sigma E-7889). Stock conc—0.5M
siRNAs: All siRNAs were obtained from Dharmacon.

### REFERENCES

(1) Bannister A J, Schneider R, Kouzarides T. Histone methylation: dynamic or static?Cell. 2002 Jun. 28; 109(7):801-6.
(2) Shi Y, Lan F, Matson C, Mulligan P, Whetstine J R, Cole P A, Casero R A, Shi Y. Histone demethylation mediated by the nuclear amine oxidase homolog LSD1. Cell. 2004 Dec. 29; 119(7):941-53.
(3) Tsukada Y, Fang J, Erdjument-Bromage H, Warren M E, Borchers C H, Tempst P, Zhang Y. Histone demethylation by a family of JmjC domain-containing proteins. Nature. 2006 Feb. 16; 439(7078):811-6.
(4) Klose R J, Kallin E M, Zhang Y. JmjC-domain-containing proteins and histone demethylation. Nat Rev Genet. 2006 September; 7(9):715-27.
(5) De Santa F, Totaro M G, Prosperini E, Notarbartolo S, Testa G, Natoli G. The histone H3 lysine-27 demethylase Jmjd3 links inflammation to inhibition of polycomb-mediated gene silencing. Cell. 2007 Sep. 21; 130(6):1083-94.
(6) Ishii M, Wen H, Corsa C A, Liu T, Coelho A L, Allen R M, Carson W F 4th, Cavassani K A, Li X, Lukacs N W, Hogaboam C M, Dou Y, Kunkel S L. Epigenetic regulation of the alternatively activated macrophage phenotype. Blood. 2009 Oct. 8; 114(15):3244-54.
(7) De Santa F, Narang V, Yap Z H, Tusi B K, Burgold T, Austenaa L, Bucci G, Caganova M, Notarbartolo S, Casola S, Testa G, Sung W K, Wei C L, Natoli G. Jmjd3 contributes to the control of gene expression in LPS-activated macrophages. EMBO J. Published online 2009 Sep. 24. doi: 10.1038/emboj.2009.271.
(8) Ivanov I I, McKenzie B S, Zhou L, Tadokoro C E, Lepelley A, Lafille J J, Cua D J, Littman D R. The orphan nuclear receptor RORgammat directs the differentiation program of proinflammatory IL-17+ T helper cells. Cell. 2006 Sep. 22; 126(6):1121-33.
(9) Wolters N M, MacKeigan J P. From sequence to function: using RNAi to elucidate mechanisms of human disease. Cell Death Differ. 2008 May; 15(5):809-19.
(10) Miossec P, Korn T, Kuchroo V K. Interleukin-17 and type 17 helper T cells. N Engl J. Med. 2009 Aug. 27; 361(9):888-98.
(11) Dardalhon V, Korn T, Kuchroo V K, Anderson A C. Role of Th1 and Th17 cells in organ-specific autoimmunity. J. Autoimmun. 2008 November; 31(3):252-6.

1. A method for identifying compounds that will be useful in treating autoimmune and inflammatory diseases or conditions comprising the step of determining whether the compound inhibits or the step of determining whether the compound activates JMJD2E.
